(19) United States
(12) Patent Application Publication
(10) Pub. No.: US 2024/0115637 A1
(43) Pub. Date: Apr. 11, 2024

(54) INFLAMMATION AND PAIN REDUCING THERAPEUTIC COMPOSITION

(71) Applicant: Glenn Edward Adams, Williamsville, NY (US)

(72) Inventor: Glenn Edward Adams, Williamsville, NY (US)

(21) Appl. No.: 18/142,289

(22) Filed: May 2, 2023

Related U.S. Application Data
(60) Provisional application No. 63/473,089, filed on May 2, 2022.

(51) Int. Cl. A61K 36/07; A61K 31/00; A61K 35/644

(52) U.S. Cl. CPC A61K 36/07; A61K 31/658; A61K 35/644

(57) ABSTRACT

A natural based therapeutic compound which helps alleviate inflammation, pain, and/or discomfort caused by disease and injuries, as well as can be used in palliative and/or hospice care includes honey, THC-9 distillate, and powdered Psilocybe cubensis.

### PRIORITY INFORMATION

The present application claims priority, under 35 U.S.C. § 119(e), from U.S. Provisional Patent Application, Ser. No. 63/473,089, filed on May 2, 2022. The entire content of U.S. Provisional Patent Application, Ser. No. 63/473,089, filed on May 2, 2022, is hereby incorporated by reference.

### BACKGROUND

Conventionally, there have been many pharmaceutical based therapeutic products developed to address inflammation, pain, and/or discomfort caused by disease and injuries, as well as natural and chemical therapeutics to be used in palliative and/or hospice care.

These conventional pharmaceutical based therapeutic products have met with varying degrees of success. Moreover, given the nature of the therapeutic products being pharmaceutical based, the conventional pharmaceutical based therapeutic products have caused many undesirable and harmful side effects.

It is, therefore, desirable to provide a natural based therapeutic compound which helps alleviate inflammation, pain, and/or discomfort caused by disease and injuries, as well as can be used in palliative and/or hospice care.

It is further desirable to provide a natural based therapeutic compound which has no or minimal undesirable side effects.

Additionally, it is desirable to provide a natural based therapeutic compound which helps alleviate inflammation, pain, and/or discomfort caused by disease and injuries, as well as can be used in palliative and/or hospice care, which has no or minimal undesirable side effects

### DETAILED DESCRIPTION

The following is a detailed description of the preferred embodiments of an inflammation and pain reducing therapeutic composition.

As mentioned above, it is desirable to provide a natural based therapeutic compound which helps alleviate inflammation, pain, and/or discomfort caused by disease and injuries, as well as can be used in palliative and/or hospice care.

With respect to the natural based therapeutic compound, the compound is a composition of an insect produced product (bee honey), a plant (cannabis), and a fungi (Psilocybe cubensis).

The honey is easily digestible and is a natural anti-inflammatory.

The preferred honey is produced by Apis millifera (the western Honeybee). The honey is filtered. Preferably, the honey is locally source so that the natural benefits of the honey can be maximized. In other words, the honey should be sourced near the area that the natural based therapeutic compound is being distributed.

The preferred form of cannabis in the natural based therapeutic compound is a THC-9 distillate, being a semi-viscous liquid between 68° F. and 72° F.

The preferred strains of Psilocybe cubensis in the natural based therapeutic compound are Yeti, Amazon-PE, and/or Malabar.

The cannabis (cannabinoids) and Psilocybe cubensis are psycho-active compounds that cause an exponential effect on each other, while also moderating the highs and lows of each other.

The cannabinoid helps to magnify effectiveness of the Psilocybe cubensis.

In other words, the THC-9 distillate activates first, providing a calming effect to the digestive tract and preventing nervous uplift of Psilocybe cubensis activation. The Psilocybe cubensis activates after about 30-45 minutes, peaking in effectiveness at about 1-2 hours, and a plateau of relief for 5-6 hours.

The ranges for the various components of the composition of the natural based therapeutic compound, by weight, are:
Honey—95% to 99.5%
THC-9 distillate—0.03% to 0.035%
Powdered Psilocybe cubensis—0.84% to 3%

The preferred composition of the natural based therapeutic compound, by weight is:
Honey—99.13%
THC-9 distillate—0.03%
Powdered Psilocybe cubensis —0.84%

The natural based therapeutic compound is made by heating/stirring the honey to about 85° F. The THC-9 distillate is added to the heated honey and the mixture is stirred and heated for about 4 hours to ensure thorough distribution of the THC-9 distillate within the honey.

The Psilocybe cubensis is grounded into a powder and stirred into the honey/THC-9 distillate mixture. This mixture is stirred and heated for an amount of time to ensure thorough distribution of the powdered Psilocybe cubensis within the honey/THC-9 distillate mixture.

It is noted that the THC-9 distillate and the powdered Psilocybe cubensis can be added to the heated honey at the same time and the mixture is stirred and heated for about 4 hours to ensure thorough distribution of the THC-9 distillate and powdered Psilocybe cubensis within the honey.

It is noted that a large mass (larger than the amount needed to make a batch of the compound) of Psilocybe cubensis to homogenize any variation in potency. The amount of Psilocybe cubensis needed to make the desired batch is taken from this larger batch of powdered Psilocybe cubensis.

The natural based therapeutic compound is an easily digestible liquid that can be orally given to users.

It is noted that the natural based therapeutic compound can be encapsulated in capsules for oral administration or mixed with a gelatin to create a soft chewable pill for oral administration.

The natural based therapeutic compound can be orally administrated to an individual to help alleviate inflammation, pain, and/or discomfort caused by disease and injuries.

Moreover, the natural based therapeutic compound can be orally administrated to an individual, as part of a palliative or hospice program help alleviate inflammation, pain, and/or discomfort.

A therapeutic compound for alleviating inflammation, pain, and/or discomfort, comprises honey; THC-9 distillate; and powdered Psilocybe cubensis.

The composition of the therapeutic compound may be 95% to 99.5% honey by weight, 0.03% to 0.035% THC-9 distillate by weight, and 0.84% to 3% powdered Psilocybe cubensis by weight.

The composition of the therapeutic compound may be s 99.13% honey by weight, 0.03% THC-9 distillate by weight, and 0.84% powdered Psilocybe cubensis by weight.

It will be appreciated that variations of the above-disclosed embodiments and other features and functions, or alternatives thereof, may be desirably combined into many other different applications.

Also, various presently unforeseen or unanticipated alternatives, modifications, variations, or improvements therein may be subsequently made by those skilled in the art which are also intended to be encompassed by the description above and the following claims.

What is claimed is:

1. A therapeutic compound for alleviating inflammation, pain, and/or discomfort, comprising:
honey;
THC-9 distillate; and
powdered Psilocybe cubensis.

2. The therapeutic compound of claim 1 wherein the composition is 95% to 99.5% honey by weight, 0.03% to 0.035% THC-9 distillate by weight, and 0.84% to 3% powdered Psilocybe cubensis by weight.

3. The therapeutic compound of claim 1 wherein the composition is 99.13% honey by weight, 0.03% THC-9 distillate by weight, and 0.84% powdered Psilocybe cubensis by weight.
